(19) United States
(12) Patent Application Publication
(10) Pub. No.: US 2020/0305851 A1
(43) Pub. Date: Oct. 1, 2020

(54) INTRAUTERINE SAMPLE COLLECTION DEVICE AND METHOD OF USING SAME

(71) Applicant: THE ROYAL INSTITUTION FOR THE ADVANCEMENT OF LEARNING/MCGILL UNIVERSITY, Montreal (CA)

(72) Inventor: Lucy Gilbert, Westmount (CA)

(21) Appl. No.: 16/651,953

(22) PCT Filed: Sep. 18, 2018
(86) PCT No.: PCT/IB2018/057191

§ 371 Date: Mar. 27, 2020

Related U.S. Application Data
(60) Provisional application No. 62/565,525, filed on Sep. 29, 2017.

(51) Int. Cl. A61B 10/02; A61B 10/00

(52) U.S. Cl. CPC A61B 10/0291; A61B 2010/0216; A61B 10/0096; A61B 10/0283

(57) ABSTRACT

A device (10) for collecting a sample from a uterus (14). The device includes a core (24) defining a core outer surface (28) and a distal brush section (30) provided with brush bristles (32) extending from the core outer surface (28). A sheath (26) receives at least part of the core (24) thereinto and defines axially opposed sheath proximal and distal ends (34 and 36). The core (24) and sheath (26) are movable relative to each other between a device retracted configuration and a device expanded configuration, wherein, in the device retracted configuration, the brush bristles (32) are compressively contained within the sheath (26) in a brush bristle compressed configuration, and, in the device expanded configuration, the brush bristles (32) are outside the sheath (26), distally to the sheath distal end (36), in a brush bristle expanded configuration wherein the brush bristles (32) span a larger volume than in brush bristle compressed configuration. Also, a kit including the device and methods of using the device.

### FIELD OF THE INVENTION

The present invention relates to general field of biological sample collection. More specifically, the present invention is concerned with intrauterine sample collection devices and methods of using same.

### BACKGROUND

Endometrial and ovarian cancers are most of the time diagnosed only when apparent symptoms lead a patient to consult a gynecologist. By that time, the cancer has most of the time entered a late stage and may prove to be difficult or impossible to eradicate. Screening patients regularly using intrauterine samples would help in early diagnosis. However, sample collection efficient enough to collect enough biological material for screening purposes using currently existing systems and devices is extremely uncomfortable to the patient unless performed under anesthesia and is therefore not routinely performed.

Against this background, there exists a need in the industry to provide novel intrauterine sample collection devices and methods. An object of the present invention is therefore to provide such improved devices and methods.

### SUMMARY OF THE INVENTION

In a broad aspect, there is provided a device for collecting a biological sample from a uterus having a uterine wall delimiting a uterine cavity, the device comprising: a substantially elongated core defining a core outer surface, the core defining a distal brush section, the brush section being provided with brush bristles extending from the core outer surface; and a substantially elongated sheath receiving at least part of the core thereinto, the sheath defining axially opposed sheath proximal and distal ends. The core and sheath are movable relative to each other between a device retracted configuration and a device expanded configuration, wherein, in the device retracted configuration, the brush bristles are compressively contained within the sheath in a brush bristle compressed configuration, and, in the device expanded configuration, the brush bristles are outside the sheath, distally to the sheath distal end, in a brush bristle expanded configuration wherein the brush bristles span a larger volume than in brush bristle compressed configuration.

There may also be provided a device wherein the brush bristles are configured and sized so that in the brush bristle expanded configuration, the brush bristles conform to a shape of at least part of the uterine cavity so that the brush bristles contact the uterine wall along a circumference thereof.

There may also be provided a device wherein in the brush bristle expanded configuration, the brush bristles span a volume that has at least a portion thereof that tapers proximally.

There may also be provided a device wherein in the brush bristle expanded configuration, the brush bristles span a volume that tapers proximally.

There may also be provided a device wherein in the brush bristle expanded configuration, the brush bristles span a substantially frusto-conical shape.

There may also be provided a device wherein the frusto-conical shape has an opening angle of between about 60 degrees and about 120 degrees.

There may also be provided a device wherein the brush bristles have a length comprised between a minimal length and a maximal length, the minimal length being between about 2 mm and about 5 mm and the maximal length being between about 10 mm and about 25 mm.

There may also be provided a device wherein the brush bristles extend from a section of the core that is about 10 mm to about 25 mm long.

There may also be provided a device wherein the brush bristles are configured and sized to exert a maximal shear stress of 10 Pa or less on the uterine wall in use.

There may also be provided a device wherein the brush bristles are configured and sized to exert a maximal shear force of 1 N or less on the uterine wall in use.

There may also be provided a device wherein, in the brush bristle expanded configuration, the brush bristles expand substantially radially outwardly from the core outer surface.

There may also be provided a device wherein the core defines an inner passageway extending axially therealong and at least one aperture extending between the core outer surface and the inner passageway in the brush section, the inner passageway being proximally in fluid communication with a vacuum device usable to create a pressure drop in the inner passageway.

There may also be provided a device wherein the vacuum device includes a syringe.

There may also be provided a device wherein the sheath includes an axially collapsible section movable between collapsible section expanded and retracted configuration, wherein the sheath is shorter in the collapsible section retracted configuration than in the collapsible section expanded configuration.

There may also be provided a device wherein the core and the sheath are both proximally mounted to a base so that in the collapsible section retracted configuration, the core and sheath are in the device expanded configuration, and in the collapsible section expanded configuration, the core and sheath are in the device retracted configuration.

There may also be provided a device wherein the core further defines a tip section distal to the brush section, the tip section being atraumatic.

There may also be provided a device wherein the tip section has a diameter smaller than a diameter of the core in the brush section.

There may also be provided a device wherein the tip section tapers in a distally leading direction.

There may also be provided a device wherein the tip section is more flexible than the brush section.

There may also be provided a device wherein at least one of the brush bristles tapers in a direction leading away from the core outer surface.

There may also be provided a device wherein at least one of the brush bristles tapers in a direction leading towards the core outer surface.

There may also be provided a device wherein at least one of the brush bristles has a transversal cross-sectional configuration selected from the group consisting of a square, a round, a triangular, a polygonal and a ring configuration.

There may also be provided a device wherein at least one of the brush bristles is substantially rectilinear in the expanded configuration.

There may also be provided a device wherein at least one of the brush bristles is substantially curved in the expanded configuration.

There may also be provided a device wherein at least one of the brush bristles is substantially jagged in the expanded configuration.

In another broad aspect, there is provided a kit including the device as defined above, a collection container and a lid.

There may also be provided a kit wherein the collection container contains a cell preserving fluid.

There may also be provided a kit wherein the cell preserving fluid is/includes at least one of Tris-EDTA, 75% ethanol, ThinPrep™ and methanol.

There may also be provided a kit wherein the lid is selectively screwable to the container at a top end thereof to close the container and at a bottom end thereof to provide a base for supporting the container, the lid being wider than the container.

In yet another broad aspect, there is provided a method of using a sheath and a brush having brush bristles for collecting a biological sample from patient having a vagina leading to a uterus having an uterine wall delimiting a uterine cavity through a cervix having a cervical canal, the brush being receivable in the sheath and slidable therealong, the method comprising: with the brush retracted in the sheath, inserting the sheath into the vagina until the sheath abuts against the cervix; while keeping the sheath abutted against the cervix, push the brush out of the sheath so that the brush enters the cervical canal and then the uterine cavity with the brush conforming to the shape of at least a portion of the uterine cavity; rotate the brush axially to collect uterine cells on the brush; withdraw the brush back into the sheath; and withdraw the sheath from the vagina.

There may also be provided a method further comprising, after having withdrawn the sheath from the vagina, plunging the brush in a collection fluid to collect at least part of the uterine cells.

There may also be provided a method wherein the brush defines an inner passageway and at least one aperture leading into the inner passageway, the method further comprising aspiring intrauterine fluids in the inner passageway through the aperture.

There may also be provided a method wherein the intrauterine fluids are undiluted biological fluids.

There may also be provided a method further comprising transferring at least part of the intrauterine fluids to the collection fluid.

In yet another broad aspect, there is provided a method of diagnosing cancer in a patient, the method comprising: collecting a biological sample as defined above; collecting a germ line sample from the patient; using next-generation sequencing methods on the germ line sample and the biological sample to identify somatic mutations in the biological sample; diagnose the patient as having cancer or as having only benign mutations at least in part on a basis of the somatic mutations.

There may also be provided a method wherein the germ line sample includes blood cells.

There may also be provided a method wherein the germ line sample includes buccal endothelial cells.

Advantageously, in some embodiments, the proposed device is relatively atraumatic and causes minimal discomfort to the patient in use. This allows its use in asymptomatic patients for screening purposes, for example. Additionally, the proposed device is, in some embodiments, relatively inexpensive to manufacture and relatively easy to use in an ergonomic manner.

The present application claims priority from U.S. Provisional patent applications 62/565,525 filed 29 Sep. 2017, the contents of which is hereby incorporated by reference in its entirety.

Other objects, advantages and features of the present invention will become more apparent upon reading of the following non-restrictive description of preferred embodiments thereof, given by way of example only with reference to the accompanying drawings.

### BRIEF DESCRIPTION FOR DRAWINGS

In the appended drawings:

FIG. 1, in a side view, illustrates an embodiment of a device for collecting an intrauterine sample, here shown in a step of a method of using the device;

FIG. 2A, in a side view, illustrates a proximal part of the device of FIG. 1 in a device retracted configuration;

FIG. 2B, in a side view, illustrates a distal part of the device of FIG. 1 in the device retracted configuration;

FIG. 3A, in a side view, illustrates a proximal part of the device of FIG. 1 in a device expanded configuration;

FIG. 3B, in a side view, illustrates a distal part of the device of FIG. 1 in the device expanded configuration;

FIG. 4A, in a side view, illustrates a proximal part of the device of FIG. 1 in a device intermediate configuration;

FIG. 4B, in a side view, illustrates a distal part of the device of FIG. 1 in the device intermediate configuration;

FIG. 5, in a side view, illustrates an other step in the use of the device of FIGS. 1 to 4B;

FIG. 6, in a side view, illustrates yet an other step in the use of the device of FIGS. 1 to 4B;

FIG. 7, in a partial perspective view, illustrates a kit including a container, a lid part and the device of FIGS. 1 to 4B;

FIG. 8, in a side cross-sectional view, illustrates the kit of FIG. 7;

FIG. 9A, in a side view, illustrates an embodiment of a brush bristle part of the device of FIGS. 1 to 4B;

FIG. 9B, in a side view, illustrates an alternative embodiment of a brush bristle part of the device of FIGS. 1 to 4B;

FIG. 9C, in a side view, illustrates an other alternative embodiment of a brush bristle part of the device of FIGS. 1 to 4B;

FIG. 10A, in a side view, illustrates yet an other alternative embodiment of a brush bristle part of the device of FIGS. 1 to 4B;

FIG. 10B, in a side view, illustrates yet an other embodiment of a brush bristle part of the device of FIGS. 1 to 4B;

FIG. 11A, in a transversal cross-sectional view, illustrates yet an other alternative embodiment of a brush bristle part of the device of FIGS. 1 to 4B;

FIG. 11B, in a transversal cross-sectional view, illustrates yet an other alternative embodiment of a brush bristle part of the device of FIGS. 1 to 4B;

FIG. 11C, in a transversal cross-sectional view, illustrates yet an other alternative embodiment of a brush bristle part of the device of FIGS. 1 to 4B;

FIG. 11D, in a transversal cross-sectional view, illustrates yet an other alternative embodiment of a brush bristle part of the device of FIGS. 1 to 4B;

FIG. 11E, in a transversal cross-sectional view, illustrates yet an other alternative embodiment of a brush bristle part of the device of FIGS. 1 to 4B; and

FIG. 11F, in a transversal cross-sectional view, illustrates yet an other alternative embodiment of a brush bristle part of the device of FIGS. 1 to 4B.

### DETAILED DESCRIPTION

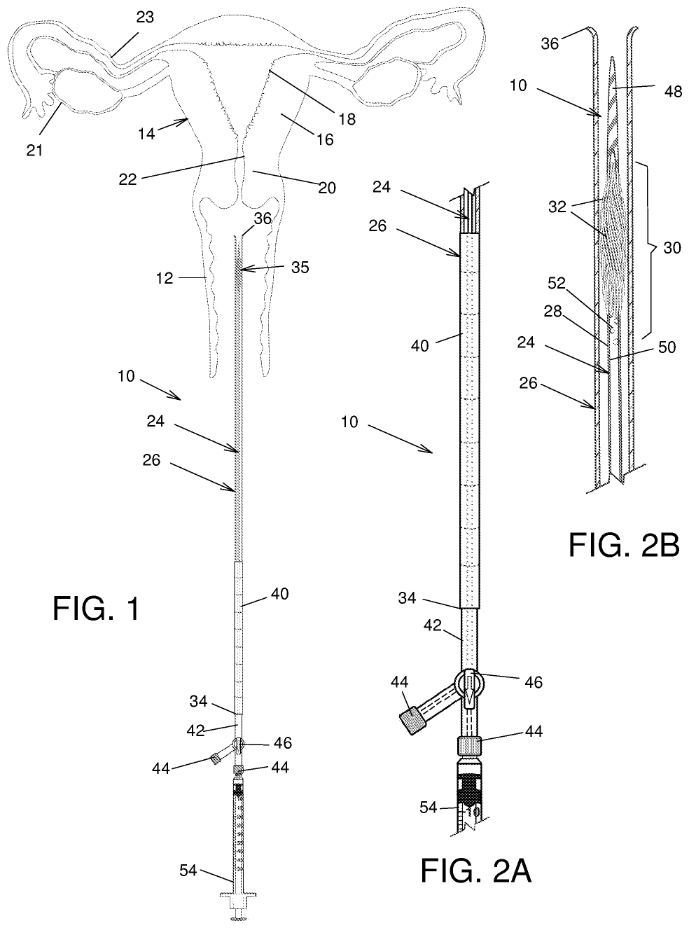

With reference to FIG. 1, there is shown a device 10 for collecting a biological sample from a patient having a vagina 12 leading to a uterus 14 having an uterine wall 16 delimiting a uterine cavity 18 through a cervix 20 having a cervical canal 22. The device 10 is here shown inserted in the vagina 12, prior to actual insertion of part thereof in the uterine cavity 18. The device 10 includes a substantially elongated core 24 and a substantially elongated sheath 26 receiving at least part of the core 24 thereinto and defining axially opposed sheath proximal and distal ends 34 and 36. As seen for example in FIG. 2B, the core 24 defines a core outer surface 28. The core 24 also defines a distal brush section 30. The brush section 30 is provided with brush bristles 32 extending from the core outer surface 28.

In the present document, the terminology distal and proximal refers to the location relative to a physician (not shown in the drawings) using the device 10. Distal elements are closer to the uterine cavity 18 in use, while proximal elements are closer to the physician. Also, the terminology “substantially” and “about” is used to denote variations in the thus qualified terms that have no significant effect on the principle of operation of the device 10. These variations may be minor variations in design or variations due to mechanical tolerances in manufacturing and use of the device 10. These variations are to be seen with the eye of the reader skilled in the art.

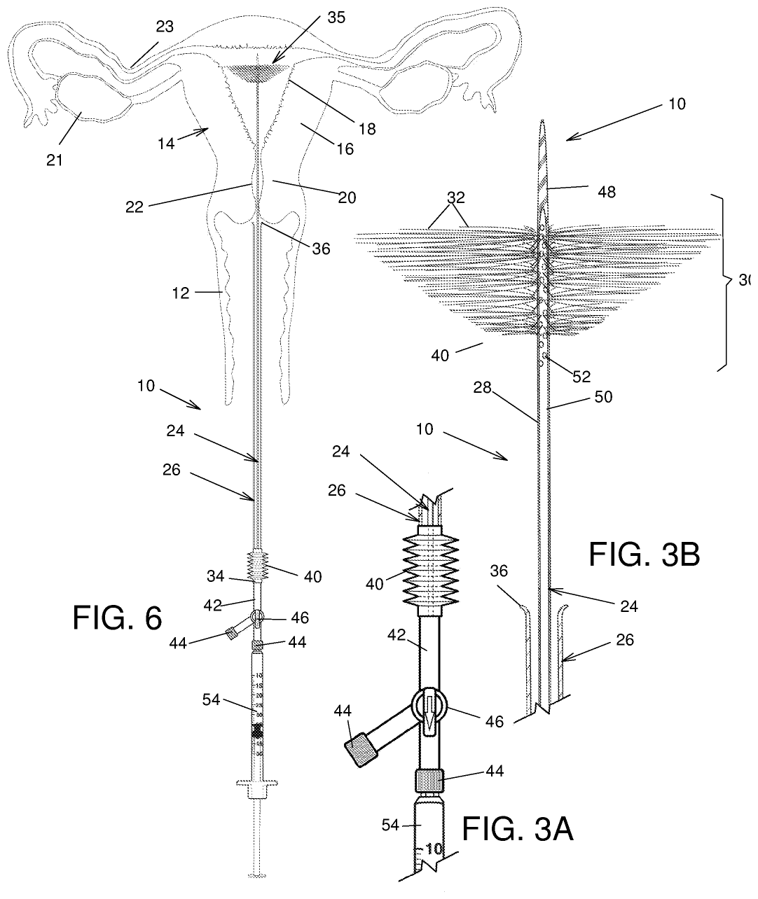

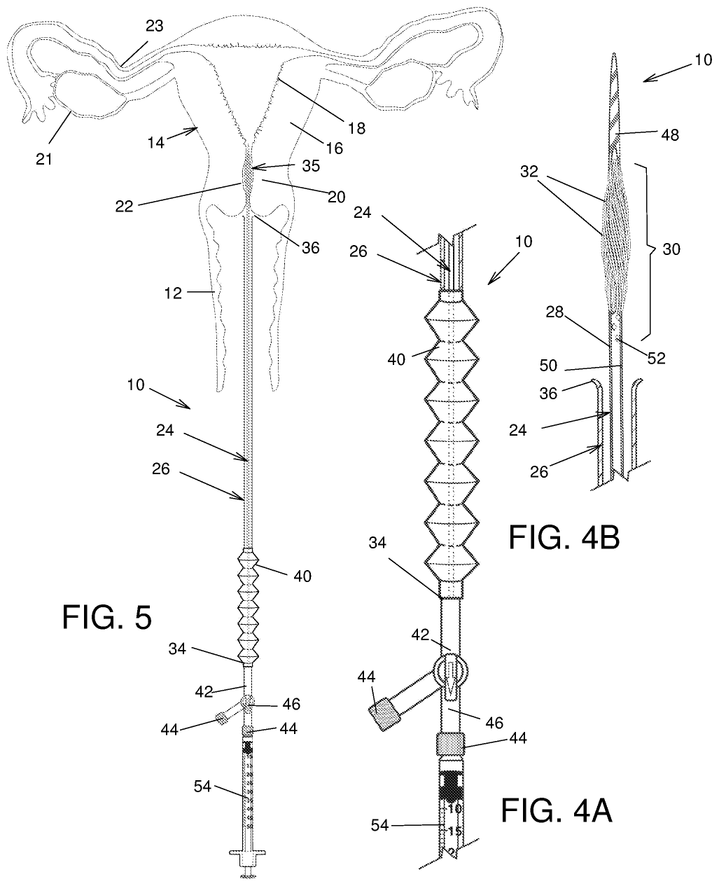

The core 24 and sheath 26 are movable relative to each other between a device retracted configuration (seen in FIGS. 1, 2A and 2B) and a device expanded configuration (seen in FIGS. 6, 3A and 3B). FIGS. 5, 4A and 4B illustrate the device 10 in a device intermediate configuration, achieved when transitioning between the device expanded and retracted configurations. Referring to FIG. 2B, in the device retracted configuration, the brush bristles 32 are compressively contained within the sheath 26 in a brush bristle compressed configuration. Referring to FIG. 3B, in the device expanded configuration, the brush bristles 32 are outside the sheath 26, distally to the sheath distal end 36, in a brush bristle expanded configuration wherein the brush bristles 32 span a larger volume than in brush bristle compressed configuration.

The sheath 26 is substantially elongated and typically tubular. In some embodiments, the sheath 26 is flexible at least in its distal portion, along with a corresponding portion of the core 24, to accommodate in use the typically curved shape of the human vagina 12. In some embodiments, the sheath 26 is substantially chalice-shaped and firm at the sheath distal end 36. In other words, the sheath expands in diameter at the sheath distal end 36 and presents a smooth curved surface to the cervix 20 to improve patient comfort. The wider sheath distal end 36 helps in collecting any adjacent leaked intrauterine fluids.

While the core 24 and the sheath 26 could in some embodiments be of constant length and simply axially slidable relative to each other, in other embodiments, as shown in the drawings, the sheath 26 includes an axially collapsible section 40 movable between collapsible section expanded and retracted configuration, shown respectively in FIGS. 2A and 3A. The sheath 26 is shorter in the collapsible section retracted configuration than in the collapsible section expanded configuration. For example, the axially collapsible section 40 includes a flexible section of the sheath 26 that includes preformed folds allowing the axially collapsible section 40 to retract and expand in an accordion-like manner.

In some embodiments, the core 24 and the sheath 26 are both proximally fixedly mounted to a base 42 so that in the collapsible section retracted configuration, the core 24 and sheath 26 are in the device expanded configuration, and in the collapsible section expanded configuration, the core 24 and sheath 26 are in the device retracted configuration. In such embodiments, insertion and withdrawal of the device 10 in the vagina 12 is decoupled from the expansion of the device 10 to expose the brush bristles 32 and retraction of the device 10 to withdraw the brush bristles 32 in the sheath 26, which facilitates use of the device 10. The base 42 is typically substantially rigid and may then serve as a handle for the device 10.

In some embodiments, the base 42 is hollow and includes at least one Luer connector 44, the purpose of which is described in further details hereinbelow. The base 42 may include two or more Luer connectors 44 selectable for fluid communication with the remainder of the device 10 using a valve 46.

With reference to FIG. 6, in a specific embodiment, the brush bristles 32 are configured and sized so that in the brush bristle expanded configuration, the brush bristles 32 conform to a shape of at least part of the uterine cavity 18 so that the brush bristles 32 contact the uterine wall 16 along a circumference thereof. It may be the case that the brush bristle expanded configuration, the brush bristles 32 can only conform to part of the uterine cavity 18, as shown in FIG. 6 where the brush would conform to the portion of the uterine cavity 18 that is adjacent the cervix 20. In other embodiments, the brush bristles 32 are sized to contact the uterine wall 16 along most of the circumference volume spanned by the brush bristles 32. It should be notes that FIG. 6 is schematic in that in many women, the uterus 14 is collapsed and does not define a large uterine cavity 18 as shown in FIG. 6.

In such configuration, the brush bristles 32 can therefore easily contact a large surface area of the uterine wall 16 to facilitate collection of a relatively large number of cells therefrom. This is to be contrasted to, for example, to the endometrial pipelle used for biopsies, which is much smaller than the uterine cavity 18. The large contact area between the brush bristles 32 and the uterine wall 16 helps in sampling simultaneously cells from diverse portions of the endometrium. Also, this large contact area allows collection of a relatively large number of cells without inflicting trauma to the endometrium. Yet furthermore, since the brush bristles 32 span a large portion of the uterine cavity 18 volume, cells floating in intrauterine fluids can more easily contact the brush bristles 32 for collection. In some embodiments, the brush bristles have a composition such that cells relatively easily stick to them, or at least stick preferentially to them relative to free floating in intrauterine fluids.

In some embodiments, in the brush bristle expanded configuration, the brush bristles 32 span a volume that has at least a portion thereof that tapers proximally. For example, the brush bristles 32 span a volume that entirely tapers proximally. A non-limiting example of such a volume is a substantially frusto-conical shape. It should be noted that in other embodiments, the brush bristles 32 span any other suitable volume.

In some embodiments, initially, prior to insertion in the uterus 14, the brush bristles 32 extend proximally from the core outer surface 28 to facilitate insertion in the cervical canal 22. In some embodiments, in the brush bristle expanded configuration, the brush bristles 32 expand substantially radially outwardly from the core outer surface 28.

In a very specific and non-limiting example, the frusto-conical shape has an opening angle of between about 60 degrees and about 120 degrees, the brush bristles have a length comprised between a minimal length and a maximal length, the minimal length being between about 2 mm and about 5 mm and the maximal length being between about 10 mm and about 25 mm and the brush bristles extend from a section of the core that is about 10 to 25 mm long.

The brush bristles 32 are typically relatively flexible to minimize discomfort to the patient and reduce or eliminate the need for anaesthesia, as opposed to many current cell collection methods. In a typical embodiment, the brush bristles 32 have a stiffness (flexural strength) that is small to enable collection of cells without excessively traumatizing the uterine wall 16, but that nevertheless have the ability to engage the uterine wall 16 with enough friction to dislodge individual cells for collection. It should be noted that this is in contract with biopsy devices that need to be much more rigid as such devices intend to remove tissue samples, while preserving the tissue structure, which is not the case necessarily in the present invention. Therefore, stiffness is such that the brush bristles 32 are stiff enough to collect cells while not significantly exfoliating the uterine wall 16. The brush bristles 32 may be made of a polymer, such as non-limitingly polyvinyl chloride (PVC), polypropylene (PP), polyethylene terephthalate (PET), Nylon, polyvinylidene fluoride (PVDF), low-density polyethylene (LDPE), high-density polyethylene (HDPE), or ultra-high-molecular-weight polyethylene (UHMWPE), among others, or a metal, such as non-limitingly a Ni—Ti superelastic metal. In some embodiments, the brush bristles 32 are non-DNA containing and are therefore made of materials that are not plant or animal based. In some embodiments, the brush bristles 32 are configured and sized and have material properties such that the brush bristles 32 exert a maximum shear stress of 10 Pa or less on uterine tissues in use. In some embodiments, the brush bristles 32 are configured and sized and have material properties such that the brush bristles 32 exert a maximum shear force of 1 N or less on uterine tissues in use.

In some embodiments, the brush bristles 32 are attached to the core 24 by any assembly method such as adhesive bonding, ultrasonic welding, sintering, laser welding, radio-frequency bonding, mechanical lock or interference, among other possibilities. In other embodiments, the brush bristles 32 are manufactured using laser-cut methods from a polymer or metal tube. Other suitable manufacturing methods are also possible.

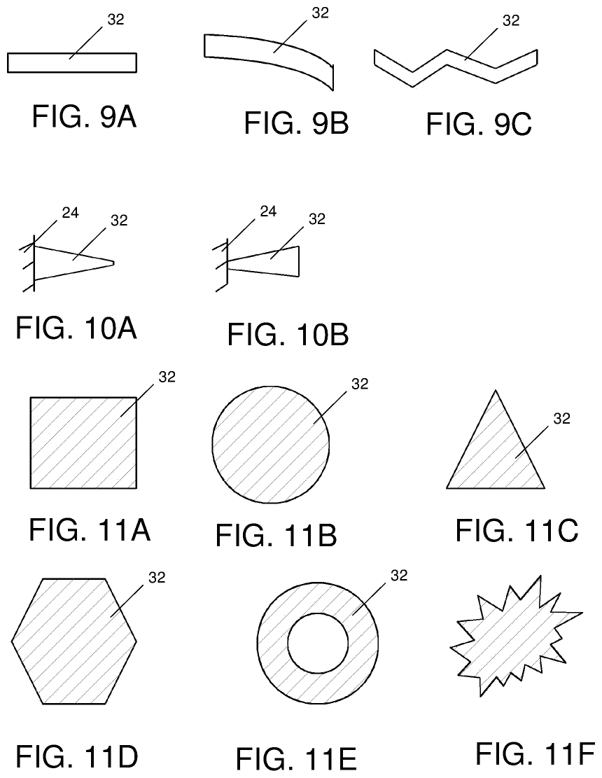

The brush bristles 32 may be in any suitable number and may have any suitable shape. All the brush bristles 32 may have the same general configuration, or brush bristles 32 of different configurations may be mixed together in the same device 10. For example, as seen respectively in FIGS. 9A, 9B and 9C, the brush bristles 32 may be respectively substantially rectilinear, substantially curved or substantially jagged in the expanded configuration, among other possibilities. Also, in another example, as seen respectively in FIGS. 9A, 10A and 10B, the brush bristles 32 may be respectively of constant diameter, taper in a direction leading away from the core 24 or taper in a direction leading towards the core 24, among other possibilities. In yet another example, as seen respectively in FIGS. 11A to 11F, the brush bristles 32 may have a transversal cross-sectional configuration selected from the group consisting of a square, a round, a triangular, a polygonal, a ring and an irregular configuration among other possibilities.

In some embodiments (not shown in the drawings), the brush section 30 terminates distally the core 24. However, as seen for example in FIG. 2B, in other embodiments, the core 24 further defines a tip section 48 distal to the brush section 30. For example, and non-limitingly, the tip section 48 is about 0.5 cm to 1.5 cm long and has a maximal diameter of about 1.5 to 2.5 mm, to terminate at a tip having a diameter of about 0.5 to 1 mm. For example, the tip section 48 is atraumatic and reduces discomfort and injury risk when the core 24 is inserted in the cervical canal 22. For example, in some embodiments, the tip section 48 has a diameter smaller than a diameter of the core 24 in the brush section 30. Therefore, in opposition to some existing devices, such as endometrial pipelles, the tip of the core 24 is not larger than other portions of the core. Typically, the tip section 48 tapers in a distally leading direction, and has for example and non-limitingly a substantially conical shape with a rounded tip. In some embodiments, the tip section 48 is more flexible than the brush section 30.

Still referring to FIG. 2B, in some embodiments, the core 24 defines an inner passageway 50 extending axially therealong and at least one aperture 52 extending between the core outer surface 28 and the inner passageway 50 in the brush section 30. Typically, a plurality of such apertures 52 are provided, which extend for example substantially radially. Having the apertures 52 in the brush section 30 ensure that the apertures 52 are spaced apart from the uterine wall 16, described below, which would be uncomfortable to the patient and could cause tissue damage. Therefore, the bush bristles 32 also act as spacers for spacing apart the apertures 52 from the uterine wall 16.

The inner passageway 50 is proximally in fluid communication with a vacuum device usable to create a pressure drop in the inner passageway 50. In a specific embodiment of the invention, the vacuum device takes the form of a syringe 54, secured to one of the Luer connectors 44. In such embodiments, the base 42 is hollow to provide a communication between the syringe 54 and the inner passageway 50. It should be noted that other vacuum devices could be used, such as a pump, among other possibilities.

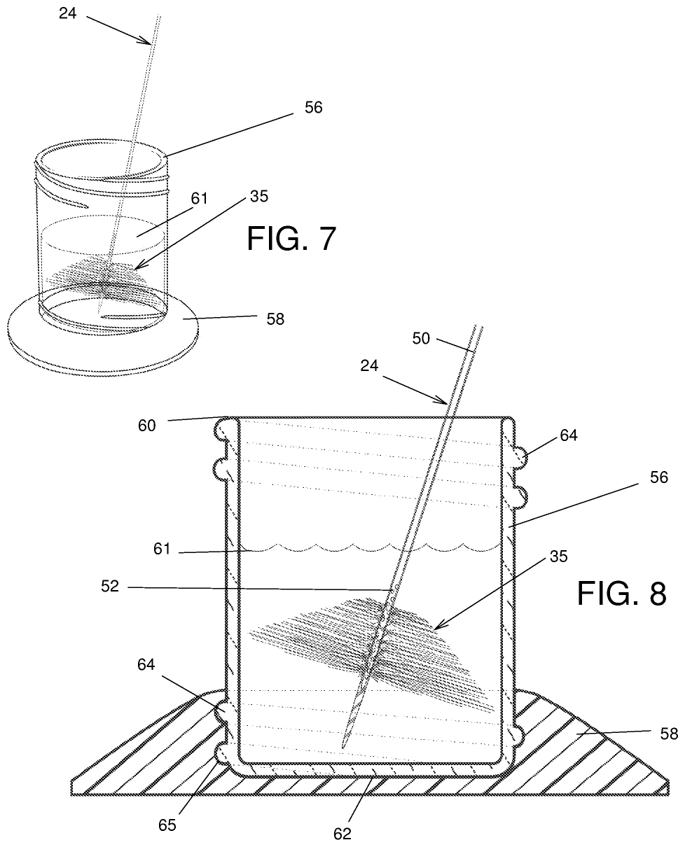

With reference to FIGS. 7 and 8, in some embodiments, the device 10 is usable with a container 56 and a lid 58. For example, the container 56 contains about 4 ml of a cell preserving fluid, but other quantities are within the scope of the invention. A non-limiting example of a suitable cell preserving fluid is a genomic DNA preserving buffer solution. The container 56 is typically deep enough to ensure the whole length of the tip section 48 and brush section 30 is submerged in the buffer.

In some embodiments, the lid 58 is selectively screwable to the container 56 at a top end 60 thereof to close the container 56 and at a bottom end 62 thereof to provide a base for supporting the container 56, the lid 58 being wider than the container 56. To that effect, the container 56 is for example substantially cylindrical and provided with external threads 64 both at the top end 60 and at the bottom end 62. The lid 58 is for example frusto-conical and provided with internal threads 65 that are configured to engage the external threads 64. The container 56 and lid 58 may conform to any suitable standard in the industry for fluid containers intended for shipping. A suitable box and label (not shown in the drawings) may be also provided so that once the container 56 has received biological material from the device 10, as described below, the closed container 56 can be shipped to laboratory for sample analysis. In some embodiments, the device 10, or portions thereof, is provided sterilized in a sealed envelope.

The core 24 and sheath 26 are typically made of any suitable medical grade material, such as plastic, polymer, or similar material. The core 24 may have for example, and non-limitingly, a maximal diameter of from about 1.5 to about 2.5 mm.

An example of a method of using the device 10 is now described referring to the sequence of FIGS. 1, 5, 6 and 7. The brush section 30, along with the brush bristles 32, are collectively referred to as a brush 35 hereinbelow. This method may also be performed using other devices similar to the device 10, but that are not identical thereto. The method is used to collect a biological sample from the uterus 14. This biological sample may include cells and, in some embodiments, intrauterine fluids. The intrauterine fluids may include cells in suspension or freely floating generic material, among other possibilities. It should be noted that while obviously uterine cells, such as endometrial cells may be collected, ovarian cells that have travelled from the ovaries 21 to the uterus 14 through the fallopian tubes 23 may also be collected in some embodiments.

The patient is typically placed in the lithotomy position, further to which a sterile, lightly lubricated vaginal speculum is employed to render the external os of the uterus 14 visible. If needed, the cervix 20 may be steadied with a tenaculum. To minimize contamination risks and maximize cell collection, in some embodiments, the biological sample is to be taken before any other intrauterine intervention. Referring to FIG. 1, with the brush 35 retracted in the sheath 26, the sheath 26 is then inserted into the vagina 12 until the sheath 26 abuts against the cervix 20. Then, as seen in FIG. 5, while keeping the sheath 26 abutted against the cervix 20, the brush 35 is pushed out of the sheath 26 so that the brush 35 enters the cervical canal 22 and then the uterine cavity 18, as seen in FIG. 6. Then, the brush 35 may conform to the shape of at least a portion of the uterine cavity 18 as the brush bristles 32 are resiliently deployed. This deployment is performed by collapsing the collapsible section 40, which effectively shortens the sheath 26 to expose the brush bristles 32. When present, the tip section 48 facilitates penetration in the cervical canal 22. Afterwards, the brush 35 is rotated axially to collect uterine cells on the brush 35, for example and non-limitingly about 360 degrees, and the brush 35 is withdrawn back into the sheath 26 so that the sheath 26 can be withdrawn from the vagina 12 while protecting the brush 35 from contamination. When the brush 35 is withdrawn back into the sheath 26, the brush bristles 32 extend distally from the core 24, as opposed to proximally prior to insertion.

When the container 56 is provided, after having withdrawn the sheath from the vagina, the brush 35 is again pushed out of the sheath 26 and the brush 35 is plunged in the collection fluid 61 to collect at least part of the uterine cells deposited on the brush bristles 32. In alternative embodiments, the brush 35 may be severed from the remainder of the core 24 and plunged in the collection fluid 61. Severance may be performed by simply cutting the brush 35 with scissors, or the brush 35 may be detached from the remainder of the core 24 by breaking a preformed weakened section of the core (not shown in the drawings).

When inner passageway 50 and syringe 54 are present, the method may also include, when the brush 35 is deployed in the uterus 14, aspiring intrauterine fluids in the inner passageway 50 through the apertures 52 by creating a suction with the syringe 54. For example, the intrauterine fluids are undiluted biological fluids. Vacuum is maintained afterwards until the intrauterine fluids may be collected in the collection fluid 61 by pressing the syringe 54 plunger. In another example, the second Luer connector 44 is used to attach a sterile fluid source thereto to provide a wash to assist in collection of the cells and intrauterine fluids. Then, the method includes pushing a sterile fluid, such as a sterile saline solution, in the uterus 14, and withdrawing, the injected fluid using the syringe 54. In such methods, the intrauterine fluids or collected sterile fluid may also be transferred to the collection fluid 61.

In some embodiments, the above collection methods allow collection of enough genetic material to diagnose cancer. In some embodiments, distinction between benign somatic mutations and malignant somatic mutations may be made. Therefore, there may be provided a method of diagnosing cancer in a patient, the method comprising collecting a biological sample as described hereinabove using the device 10 and collecting a germ line sample from the patient. Then, the method includes using next-generation sequencing (NGS) methods on the germ line sample and the biological sample to identify somatic mutations. Finally, the method includes diagnosing the patient as having cancer or as having only benign mutations on a basis of the somatic mutations. For example, the germ line sample includes blood cells. In these embodiments, collecting the germ line sample includes drawing a blood sample from the patient. In another example, the germ line sample includes buccal endothelial cells. In these embodiments, collecting the germ line sample includes performing a buccal swab or having the patient spit a saliva sample. Any other suitable germ line sample may also be used. An example of suitable genetic methods for identifying cancer is described below, but any other suitable genetic method may be used. For example, PCT application publication WO2017220782 published Dec. 28, 2017, which is hereby incorporated by reference in its entirety, describes such a method.

More specifically, as cancer cells exfoliates more easily than normal cells, and as the uterus is a continuous tract from the fallopian tube, a cytologic sample taken from the uterus is likely to have traces of cancer cells very early in the process of carcinogenesis of the ovary, fallopian tube and endometrium. In very early stage, when the tumour is very small, the biological sample will have only a very low number of cancer cells amidst large numbers of normal endometrial cells, precluding pathologic detection. To identify these small number of cancer cells, a DovEEgene™-HaloPlex™ System may be used. This system uses next-generation sequencing (NGS) of a genomic segment at very deep coverage to identify cancer mutations in a small fraction of DNA templates from cancer cells and a DNA-tagging technology to minimize errors resulting from possible PCR amplification bias and confidently identify rare variants in the <1% range.

In summary, this method attempts to identify all subtypes (type I and II) of ovarian and endometrial cancers, at an early stage, using an innovative uterine sampling system (the device 10) as well as a bespoke assay that includes gene panel design, bioinformatic analysis process and machine learning, to identify small amounts of cancer DNA in samples from inside the uterus and distinguish it from non-malignant conditions. In parallel the approach introduces the novelty of being able to identify predisposing germline mutations in the established inherited breast and ovarian cancer genes, BRCA1 and BRCA2, as well as in the predisposing genes PTEN, TP53, MLH1 and MSH2 or any other gene of prognostic value, while also simultaneously detecting somatic BRCA1 or BRCA2 mutations in the sample that can inform treatment decisions.

A high sensitivity assay has been developed to detect low frequency mutations at high sensitivity from cells collected with the uterine brush. This assay interrogates the entire coding sequence in contrast to so called “hotspots”. This increases the sensitivity in detecting somatic mutations, and covers genes for which there are no established hotspots and detects germline variants at the same time in genes such as BRCA1 and BRCA2. The DOvEEgene™-HaloPlex™ assay is based on Agilent DOvEEgene-Haloplex technology and is specially designed to be suitable for all types of samples including formaldehyde-fixed paraffin-embedded (FFPE) tissues. The panel was designed to amplify the entire exonic regions of genes known to be mutated in ovarian cancer, for example is a specific embodiment: AKT1, APC, CDKN2A, CTNNB1, FBXW7, FGFR2, KRAS, NRAS, PIK3CA, PIK3R1, PPP2R1A, PTEN, RNF43, BRCA1, BRCA2, MLH1, MSH2 and TP53 (see FIG. 3 for a description of the design). In total, a few thousand amplicons may be used. Genetic sequencing may for example be performed as follows:

### Preparation of the Samples

1 DNA extraction from saliva samples
Upon reception of saliva samples in an Oragene™ saliva collection kit, genomic DNA (gDNA) is extracted a Chemagen™ MSMI (Perkin-Elmer)
   2 DNA extraction from brush samples

Upon reception of brush samples in ThinPrep™ solution, or in genomic DNA preserving buffer, genomic DNA (gDNA) is extracted using a Chemagen™ MSMI (Perkin-Elmer)

3 DNA quantification
Quantification is performed with PicoGreen™ using a Janus liquid handler and a Tecan Spark 10M plate reader
   4 DNA integrity verification
Sample integrity and fragment length is verified by running samples on a 1% precast gel

### Sample Normalization for Subsequent HaloPlex^{HS} Capture

5 Normalization of saliva and brush samples
gDNA concentrations between 50 and 550 ng are required. Samples are thus either diluted using the JANUS Varispan Automation Workstation™ (PerkinElmer) or concentrated by hand using NucleoMag™ NGS Clean-up and Size Select beads, based on their concentration as measured previously with PicoGreen™.

### Automated Sample Capture Using HaloPlex^{HS}™ on the NGS BRAVO™ Workstation

6 Samples are captured using the HaloPlex^{HS} according to a customized protocol for automisation on the NGS BRAVO workstation (see Annex for further details).
6.1. The normalized gDNA from the previous steps are digested with restriction enzymes.
6.2. The digested DNA is hybridized to the HaloPlex^{HS} probe library.
6.3. The circularized DNA hybrids are purified and ligated.
6.4. The target DNA is captured and washed.
6.5. The captured target library is amplified by PCR.
6.6. The amplified target DNA is purified.
6.7. The enriched target DNA is validated and quantified.
6.8. Validated and quantified samples are pooled for multiplexed sequencing.

### Sequencing on the Illumina HiSeq 2500

7 Pooled samples are sequenced using 100 bp or 125 bp paired-end reads according to Agilent guidelines for HaloPlex^{HS}.

In brief, the targeted capture method is specifically designed to identify low allele frequency variants through the attachment of a 10 nucleotide-long molecular barcode to the captured sample DNA molecules. Typical sensitivity achieved is in the region of 0.4-0.5%, but can detect variants below 0.1%. Confidence in identifying a mutation correctly can be achieved by designing different but overlapping “probes” that are used to capture and analyze different DNA strands that include the DNA sequence of interest. In addition, these probes are designed to be able to target complementary DNA strands marked as “sense” or “antisense”, with both strands being captured in a very large fraction (for example over 99%) of the target. This allows a novel way to analyze the data: during downstream analysis of the sequencing data, molecular barcode sequence data are used to collapse reads originating from the same sample molecule, but also by sequencing the same base from complementary DNA molecules, which improves base calling accuracy by removing artifacts and allows for accurate quantification of the mutant allele fraction within each sample.

After sequencing, various bioinformatics methods may be used. For example, an analytical pipeline that is designed to combine sensitivity with specificity in order to achieve the aims of the assay. Data analysis is carried out using a bespoke pipeline utilizing initially the SureCall™ software tool (Agilent), followed by the following analysis approach. Amplicon probe identification that captures a specific fragment is used, and as we know which strand is targeted by an amplicon probe, we bioinformatically identify which DNA strand is captured and sequenced, marking it as being originally a “sense” or “antisense” DNA strand. If both sense and antisense DNA strand derived sequences agree, then the result is retained. This process is achieved by counting the number of sense and antisense strands that contained each mutation found. This information is then used to either retain or filter away mutations for increasing the specificity of the data produced.

One problem with high sensitivity sequencing is the issue of cross sample contamination, either during sample handling or during the sequencing process within the instrument, as for cost reasons, Next generation sequencing libraries need to be combined in a “multiplexed library” and sequenced as part of one sequencing reaction. The sequencing reads obtained are then assigned to the specific original library through a process involving the analysis of sample specific index sequence. However, minute contaminations are possible. Therefore, a customized variant call filtering approach that is specific for this approach may be used. It involves creating a panel of normal, germline samples which are sequenced at high coverage to create a list of germline variants for all samples to be studied. By identifying mutations that are present in the somatic sample, but not in the germ line sample, various somatic mutation parameters may be identified.

Classification of patients as having cancer or only benign mutation can be done with reference to a reference database, in which the cancer status of the patients are known, by using classification techniques. Parameters used for such classification may include, age, body mass index (BMI), total mutation burden, and presence of specific mutations, among others.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can be modified, without departing from the spirit and nature of the subject invention as defined in the appended claims.

1. A device for collecting a biological sample from a uterus having a uterine wall delimiting a uterine cavity, the device comprising:
a substantially elongated core defining a core outer surface, the core defining a distal brush section, the brush section being provided with brush bristles extending from the core outer surface; and
a substantially elongated sheath receiving at least part of the core thereinto, the sheath defining axially opposed sheath proximal and distal ends;
the core and sheath being movable relative to each other between a device retracted configuration and a device expanded configuration, wherein, in the device retracted configuration, the brush bristles are compressively contained within the sheath in a brush bristle compressed configuration, and, in the device expanded configuration, the brush bristles are outside the sheath, distally to the sheath distal end, in a brush bristle expanded configuration wherein the brush bristles span a larger volume than in brush bristle compressed configuration.

2. The device as defined in claim 1, wherein the brush bristles are configured and sized so that in the brush bristle expanded configuration, the brush bristles conform to a shape of at least part of the uterine cavity so that the brush bristles contact the uterine wall along a circumference thereof.

3. The device as defined in claim 1, wherein in the brush bristle expanded configuration, the brush bristles span a volume that has at least a portion thereof that tapers proximally.

4. (canceled)

5. The device as defined in claim 3, wherein in the brush bristle expanded configuration, the brush bristles span a substantially frusto-conical shape.

6. The device as defined in claim 5, wherein the frusto-conical shape has an opening angle of between about 60 degrees and about 120 degrees.

7. (canceled)

8. (canceled)

9. The device as defined in claim 1, wherein the brush bristles are configured and sized to exert a maximal shear stress of 10 Pa or less on the uterine wall in use.

10. The device as defined in claim 1, wherein the brush bristles are configured and sized to exert a maximal shear force of 1 N or less on the uterine wall in use.

11. The device as defined in claim 1, wherein, in the brush bristle expanded configuration, the brush bristles expand substantially radially outwardly from the core outer surface.

12. The device as defined in claim 1, wherein the core defines an inner passageway extending axially therealong and at least one aperture extending between the core outer surface and the inner passageway in the brush section, the inner passageway being proximally in fluid communication with a vacuum device usable to create a pressure drop in the inner passageway.

13. The device as defined in claim 12, wherein the vacuum device includes a syringe.

14. The device as defined in claim 1, wherein the sheath includes an axially collapsible section movable between collapsible section expanded and retracted configuration, wherein the sheath is shorter in the collapsible section retracted configuration than in the collapsible section expanded configuration.

15. The device as defined in claim 14, wherein the core and the sheath are both proximally mounted to a base so that in the collapsible section retracted configuration, the core and sheath are in the device expanded configuration, and in the collapsible section expanded configuration, the core and sheath are in the device retracted configuration.

16. The device as defined in claim 1, wherein the core further defines a tip section distal to the brush section, the tip section being atraumatic.

17. (canceled)

18. The device as defined in claim 16, wherein the tip section tapers in a distally leading direction.

19. The device as defined in claim 16,
wherein the tip section is more flexible than the brush section.

20. The device as defined in any one of claims 1 to 19, wherein at least one of the brush bristles either tapers in a direction leading away from the core outer surface or tapers in a direction leading towards the core outer surface.

21. (canceled)

22. The device as defined in claim 1, wherein at least one of the brush bristles has a transversal cross-sectional configuration selected from the group consisting of a square, a round, a triangular, a polygonal and a ring configuration.

23. The device as defined in claim 1, wherein at least one of the brush bristles is substantially rectilinear, curved or jagged in the expanded configuration.

24. (canceled)

25. (canceled)

26. A kit including the device as defined in claim 1, a collection container and a lid, wherein the collection container contains a cell preserving fluid.

27. (canceled)

28. (canceled)

29. The kit as defined in claim 26 wherein the lid is selectively screwable to the container at a top end thereof to close the container and at a bottom end thereof to provide a base for supporting the container, the lid being wider than the container.

30.-37. (canceled)
